(19) United States
(12) Patent Application Publication
(10) Pub. No.: US 2006/0185681 A1
(43) Pub. Date: Aug. 24, 2006

(54) Medical treatment position selection device, medical treatment position selection system, medical treatment position selection method, and computer program

(75) Inventors: Kazuhiko Amano, Tokyo-to (JP); Yoshito Mukaino, Fukuoka-shi (JP)

Correspondence Address: OLIFF & BERRIDGE, PLC, P.O. BOX 19928, ALEXANDRIA, VA, 22320, US

(73) Assignee: Seiko Epson Corporation, Tokyo (JP)

(21) Appl. No.: 11/340,744

(22) Filed: Jan. 27, 2006

(30) Foreign Application Priority Data
Jan. 28, 2005 (JP) 2005-021987
Jan. 28, 2005 (JP) 2005-021986
Jan. 12, 2006 (JP) 2006-004726

(51) Int. Cl. A61B 19/00

(52) U.S. Cl. 128/897

(57) ABSTRACT

A medical treatment position selection device includes a load motion instructing unit that instructs a load motion as a movement of a living body, and a medical treatment position instructing unit that instructs a medical treatment position corresponding to the load motion instructed by the load motion instructing unit.

### BACKGROUND

1. Technical Field

The present invention relates to a medical treatment position selection device, to medical treatment position selection system, to a medical treatment position selection method, and to a computer program.

2. Related Art

As for movements of a person, 200 or more joints are connected to one another, thereby allowing dynamic movements, such as fine manual movements or sports motions. However, when the person has a pain during his movement, the connection of muscles, skeleton, and joints are limited, and thus original movement ability cannot be exhibited. In order to perform an acupuncture treatment on a therapeutic point for the sake of ease of the pain, a region, which is a cause for the pain during the physical movement, needs to be specified.

A load motion is an action, which causes the pain, in a diagnosis method for specifying the region as the cause for the pain during the physical movement. The diagnosis method, in which the region as the cause for the pain is specified on the basis of the load motion, can perform a biomechanical acupuncture treatment so as to improve some abnormality in movement. Further, there are known books in which a search of points for acupuncture treatment and load motions for confirming medical treatment effect are described (for example, see ‘Meridian Test’, Yoshito Mukaino, Ishiyaku Publishers, Inc., 1999).

However, the load motion for specifying the region as the cause for the pain during the physical movement is a motion which can be recognized only by a research worker or a doctor. And then, in the related art, the acupuncture treatment using the load motions is spread by personal tradition. Due to the personal tradition, it is difficult to ensure reproducibility of a load motion instruction and reproducibility of medical treatment effect on the basis of the load motion instruction.

Further, in the related art, a curer, such as a doctor, needs to directly instruct a patient or a subject, of the load motions, and it is not easy for the people to perform the load motions. Further, in a case of training a new curer, a trainer needs to directly instruct an initiator of the load motions.

### SUMMARY

An advantage of some aspects of the invention is that it allows a load motion, which is used for specifying a medical treatment position, to be instructed with high reproducibility.

Another advantage of some aspects of the invention is that it allows a medical treatment position to be instructed according to a phenomenon, such as a pain in a living body caused by a load motion or the like.

Still another advantage of some aspects of the invention is that it allows stimulation effect in a living body on the basis of a load motion to be confirmed.

According to a first aspect of the invention, a medical treatment position selection device includes a load motion instructing unit that instructs a load motion as a movement of a living body, and a medical treatment position instructing unit that instructs a medical treatment position corresponding to the load motion instructed by the load motion instructing unit. Here, the load motion can be set movements through the connection of multiple joints and multiple axes of the living body.

According to this configuration, with the load motion instructing unit, the load motion, which is used to specify the medical treatment position, can be instructed with high reproducibility. And then, the medical treatment position instructing unit can instruct the medical treatment position to a position corresponding to a shape of the load motion executed according to the instruction of the load motion instructing unit with high reproducibility. Therefore, according to the above-described configuration, a curer, such as a doctor or the like, does not need to directly instruct a patent or a subject of the load motions, and thus the people can easily execute the load motions (for specifying the medical treatment position). Further, according to the above-described configuration, in a case of training a new curer, a trainer does not need to directly instruct an initiator of the load motions.

In the medical treatment position selection device according to the first aspect of the invention, it is preferable that the load motion instructing unit instruct plural types of load motions, and the medical treatment position instructing unit instruct medical treatment positions associated with the individual load motions.

According to this configuration, multiple load motions can be instructed with high reproducibility, and the medical treatment positions corresponding to the individual load motions can be individually instructed with high reproducibility.

The medical treatment position selection device according to the first aspect of the invention may further include an input unit that specifies one of the plural types of load motions. In this case, the medical treatment position instructing unit may instruct the medical treatment position associated with the load motion specified by the input unit.

According to this configuration, for example, as for one of plural types of load motions, when the living body executes the load motion on the basis of the instruction of the load motion instructing unit, and when a pain occurs in the living body, the medical treatment position corresponding to the specific load motion causing the pain can be instructed. Therefore, for the patient or the like, the medical treatment position can be effectively instructed.

In the medical treatment position selection device according to the first aspect of the invention, it is preferable that, when a predetermined feeling occurs in a living body due to the load motion, the input unit specify the load motion.

In the medical treatment position selection device according to the first aspect of the invention, it is preferable that the predetermined feeling be a pain.

According to the above-described configuration, for example, when the predetermined feeling, such as the pain or the like, occurs in the living body due to the load motion, the input unit is operated by the living body, such that the medical treatment position corresponding to the load motion can be instructed. And then, the living body itself treats the medical treatment position, and thus the pain can be reduced or eliminated.

The medical treatment position selection device according to the first aspect of the invention may further include a load motion type input unit that, when a predetermined feeling occurs in the living body, inputs a type of a specified load motion.

In the medical treatment position selection device according to the first aspect of the invention, it is preferable that, when the predetermined feeling occurs in the living body, the load motion type input unit selects and inputs a degree of the predetermined feeling in the living body according to the type of the specified load motion.

The medical treatment position selection device according to the first aspect of the invention may further include a load motion degree notice unit that, when a predetermined feeling occurs in the living body, digitizes a degree of a predetermined feeling in the living body according to a type of a specified load motion and notices the digitized degree of the predetermined feeling in the living body in advance.

The medical treatment position selection device according to the first aspect of the invention may further include a load motion degree input unit that, when a predetermined feeling occurs in the living body, inputs a degree of the predetermined feeling in the living body according to a type of a specified load motion.

The medical treatment position selection device according to the first aspect of the invention may further include a load motion degree selection and input unit that, when a predetermined feeling occurs in the living body, selects and inputs a degree of the predetermined feeling in the living body according to a type of a specified load motion.

The medical treatment position selection device according to the first aspect of the invention may further include a load motion type storing unit that, when a predetermined feeling occurs in the living body, stores a type of a specified load motion.

The medical treatment position selection device according to the first aspect of the invention may further include a load motion type storage data notice unit that, when the predetermined feeling occurs in the living body, reads out the type of the specified load motion from the load motion type storing unit and notices the type of the specified load motion.

The medical treatment position selection device according to the first aspect of the invention may further include a load motion degree storing unit that, when a predetermined feeling occurs in the living body, stores a degree of the predetermined feeling in the living body according to a type of a specified load motion.

The medical treatment position selection device according to the first aspect of the invention may further include a load motion degree storage data notice unit that, when the predetermined feeling occurs in the living body, reads out the degree of the specified load motion from the load motion degree storing unit and notices the degree of the specified load motion.

In the medical treatment position selection device according to the first aspect of the invention, it is preferable that, when the type of the specified load motion is read out from the load motion type storing unit and is noticed, the degree of the specified load motion be read out from the load motion degree storing unit and be noticed from the load motion degree storage data notice unit in the same unit of notice.

The medical treatment position selection device according to the first aspect of the invention may further include an identification (ID) input unit, with which a person specifies himself to select the medical treatment position. In this case, a plurality of load motion type storage data and load motion degree storage data for plural types of load motions may be input to the load motion type storing unit and a load motion degree storing unit in one unit of measurement and may be stored therein in at least one unit of measurement.

The medical treatment position selection device according to the first aspect of the invention may further include an ID input unit, with which a person specifies himself to select the medical treatment position, and a medical treatment judging unit that, when a plurality of load motion type storage data and load motion degree storage data for plural types of load motions are set as one unit of measurement, inputs load motion type storage data and load motion degree storage data to the load motion type storing unit and a load motion degree storing unit in different units of measurement, and compares load motion type storage data and load motion degree storage data in different units of measurement.

The medical treatment position selection device according to the first aspect of the invention may further include an ID input unit, with which a person specifies himself to select the medical treatment position. In this case, a plurality of load motion type storage data and load motion degree storage data for plural types of load motions may be set as one unit of measurement. Further, load motion type storage data and load motion degree storage data may be input to the load motion type storing unit and a load motion degree storing unit in different units of measurement, and load motion type storage data and load motion degree storage data may be individually multiplied in different units of measurement.

The medical treatment position selection device according to the first aspect of the invention may further include an ID input unit, with which a person specifies himself to select the medical treatment position. In this case, a plurality of load motion type storage data and load motion degree storage data for plural types of load motions may be set as one unit of measurement. Further, load motion type storage data and load motion degree storage data may be input to the load motion type storing unit and a load motion degree storing unit in different units of measurement, and a sequence of the medical treatment positions associated with the individual load motions may be stored.

The medical treatment position selection device according to the first aspect of the invention may further include an ID input unit, with which a person specifies himself to select the medical treatment position. In this case, a plurality of load motion type storage data and load motion degree storage data for plural types of load motions may be set as one unit of measurement. Further, load motion type storage data and load motion degree storage data may be input to the load motion type storing unit and a load motion degree storing unit in different units of measurement, and a sequence of medical treatment positions associated with the individual load motions may be noticed.

In the medical treatment position selection device according to the first aspect of the invention, it is preferable that the medical treatment position instructed by the medical treatment position instructing unit be a therapeutic point.

According to this configuration, the therapeutic point, which is used in an acupuncture treatment or an acupressure treatment, can be accurately instructed according to a current state of the living body with high reproducibility.

In the medical treatment position selection device according to the first aspect of the invention, it is preferable that the medical treatment position instructing unit instruct a plurality of medical treatment positions about one load motion.

According to this configuration, a plurality of therapeutic points can be accurately instructed according to the current state of the living body with high reproducibility, and thus the living body can be effectively treated.

In the medical treatment position selection device according to the first aspect of the invention, it is preferable that the medical treatment position instructing unit instruct a medical treatment sequence of the plurality of medical treatment positions.

According to this configuration, the living body can be effectively treated.

In the medical treatment position selection device according to the first aspect of the invention, it is preferable that the medical treatment position instructing unit have a medical treatment method presenting unit that instructs a medical treatment method against the medical treatment position.

According to this configuration, according to the current state of the living body, the medical treatment method can be instructed with high reproducibility, as well as the instruction of the medical treatment position.

In the medical treatment position selection device according to the first aspect of the invention, it is preferable that the load motion instructing unit instruct the load motion by use of at least one of a motion picture, a still picture, and sound, and the medical treatment position instructing unit instruct the medical treatment position by use of at least of a motion picture, a still picture, and sound.

According to this configuration, for example, the load motion as the movement through the connection of multiple joints and multiple axes of the living body can be instructed clearly and easily to understand. Therefore, the living body can be avoided from executing an erroneous load motion, and a medical treatment can be provided with high reliability at low cost.

In the medical treatment position selection device according to the first aspect of the invention, it is preferable that the motion picture or the still picture is a three-dimensional computer graphic.

According to this configuration, the load motion can be instructed clearly and easily to understand by use of various monitors.

The medical treatment position selection device according to the first aspect of the invention may further include a measuring unit that measures the living body whose medical treatment position instructed by the medical treatment position instructing unit is stimulated or treated.

According to this configuration, it is possible to objectively evaluate whether or not a stimulation or medical treatment on the living body whose medical treatment position instructed by the medical treatment position instructing unit is stimulated or treated is effective. That is, the effect of the stimulation or the like on the living body on the basis of the load motion can be quantitatively confirmed.

In the medical treatment position selection device according to the first aspect of the invention, the measuring unit may have a left and right weight measuring unit that individually measures weights of left and right legs in the living body.

According to this configuration, it is possible to quantitatively confirm whether or not a slant (that is, lateral difference) in a basic posture of the living body is reduced by the stimulation or medical treatment on the medical treatment position instructed by the medical treatment position instructing unit.

In the medical treatment position selection device according to the first aspect of the invention, the measuring unit may have a pulse wave measuring unit that measures pulse waves of the living body.

According to this configuration, with the pulse wave, it is possible to confirm easily and accurately whether or not the state of the living body is improved by the stimulation or medical treatment on the medical treatment position instructed by the medical treatment position instructing unit.

The medical treatment position selection device according to the first aspect of the invention may further include a medical treatment result judging unit that, on the basis of the measurement result of the measuring unit, judges whether or not an abnormality is reduced in the living body whose medical treatment position is stimulated or treated.

Here, the stimulation preferably includes acupuncture, acupressure, moxibustion, or massage.

According to this configuration, the medical treatment effect of the living body can be quantitatively judged. Therefore, for example, even when a pain in the living body is eliminated, it is possible to accurately judge whether or not an additional medical treatment is necessary due to an insufficient medical treatment.

According to a second aspect of the invention, a medical treatment position selection system includes a communication terminal that is connectable to a communication line, and a medical treatment position selection device having a load motion instructing unit that instructs the communication terminal of a load motion as a movement of a living body through the communication line, an input unit that receives corresponding information output from the communication terminal, which receives the instruction of the load motion, and a medical treatment position instructing unit that instructs the communication terminal of a medical treatment position according to the corresponding information received by the input unit through the communication line. Here, the load motion can be movements through the connection of multiple joints and multiple axes of the living body.

According to this configuration, the load motion instructing unit of the medical treatment position selection device can instruct the communication terminal of the load motion, which is used to specify the medical treatment position, with high reproducibility. Accordingly, an operator (patient or the like) of the communication terminal can accurately execute the load motion with high reproducibility. And then, the communication terminal can input, to the input unit of the medical treatment position selection device, the corresponding information of whether or not the predetermined feeling occurs in the living body due to the execution of the load motion. And then, the medical treatment position instructing unit of the medical treatment position selection device can instruct the communication terminal of the medical treatment position according to the corresponding information. Accordingly, the medical treatment position instructing unit can instruct the medical treatment position at a position corresponding to the load motion executed according to the instruction of the load motion instructing unit with high reproducibility. Therefore, according to the above-described configuration, a curer, such as a doctor or the like, does not need to directly instruct the patient or subject of the load motions, and the people can easily execute the load motions (for specifying the medical treatment positions) by use of the communication terminal, such as a cellular phone. Further, according to the above-described configuration, in a case of training a new curer, a trainer does not need to directly instruct an initiator of the load motions. As a result, according to the above-described configuration, the patient himself can treat the medical treatment position specified on the basis of the reaction of the living body against the load motion with high reproducibility.

The medical treatment position selection system according to the second aspect of the invention may further include a database that holds information indicating a plurality of load motions, which are movements through the connection of multiple joints and multiple axes of the living body. In this case, the load motion instructing unit may instruct any one of the plurality of load motions according to a request of the communication terminal, and the medical treatment position instructing unit may instruct medical treatment positions correspondingly associated with the plurality of load motions.

According to this configuration, with the database, various load motions can be systematized and held. And then, with the database, multiple load motions can be instructed with high reproducibility, and the medical treatment positions corresponding to the individual load motions can be individually instructed with high reproducibility. For example, when the operator of the communication terminal executes one of plural types of load motions on the basis of the instruction of the load motion instructing unit, and when the operator has a pain, the medical treatment position corresponding to the specific load motion causing the pain can be instructed. Therefore, for the patient or the like, the medical treatment position can be effectively instructed.

In the medical treatment position selection system according to the second aspect of the invention, it is preferable that the corresponding information output from the communication terminal include information for specifying any one of the plurality of load motions.

According to this configuration, the medical treatment position corresponding to the load motion specified by the corresponding information can be instructed to the operator of the communication terminal or the like.

In the medical treatment position selection system according to the second aspect of the invention, it is preferable that the load motion instructing unit have a load motion specifying unit that, when the living body performs the load motion and a predetermined feeling occurs in the living body, obtains information for specifying the load motion, which is output from the communication terminal as the corresponding information.

According to this configuration, for example, when the predetermined feeling, such as a pain or the like, occurs due to the load motion, the communication terminal is operated by the living body, such that the medical treatment position corresponding to the load motion can be instructed. And then, the living body itself treats the instructed medical treatment position, such that the pain or the like can be reduced or eliminated.

In the medical treatment position selection system according to the second aspect of the invention, it is preferable that the medical treatment position instructed by the medical treatment position instructing unit be a therapeutic point.

According to this configuration, the therapeutic point, which is used in an acupuncture treatment, an acupressure treatment, or a moxibustion treatment, can be accurately instructed according to the current state of the living body with high reproducibility.

In the medical treatment position selection system according to the second aspect of the invention, it is preferable that the load motion instructing unit present the content representing the load motion to the communication terminal by use of at least one of a motion picture, a still picture, and sound, and the medical treatment position instructing unit present the content representing the medical treatment position to the communication terminal by use of at least one of a motion picture, a still picture, and sound.

According to this configuration, in the communication terminal, for example, with the motion picture, the load motion as the movement through the connection of multiple joints and multiple axes of the living body can be displayed clearly and easily to understand. Therefore, the living body can be avoided from executing an erroneous load motion, and a medical treatment can be provided with high reliability at low cost.

In the medical treatment position selection system according to the second aspect of the invention, it is preferable that the motion picture or the still picture be a three-dimensional computer graphic.

According to this configuration, with a monitor of the communication terminal, the load motion can be displayed clearly and easily to understand.

In the medical treatment position selection system according to the second aspect of the invention, it is preferable that the medical treatment position selection device have a fee calculating unit that calculates a fee to be charged on the basis of the number of instruction times of the load motion instructing unit or the number of instruction times of the medical treatment position instructing unit.

According to this configuration, the price according to a use amount of the medical treatment position selection system by the communication terminal can be accurately calculated, and the development of an improved medical treatment position selection system can be promoted by the price.

According to a third aspect of the invention, a medical treatment position selection method includes causing a medical treatment position instructing unit connected to a communication line to transmit information instructing a load motion as a movement of a living body, to a communication terminal through the communication line, and causing the communication terminal to display the load motion by use of a motion picture, a still picture, characters, or sound, when a predetermined feeling occurs in the living body executing the load motion, causing corresponding information, which indicates that the predetermined feeling occurs, to be input to the communication terminal, and causing the communication terminal to transmit the corresponding information to a medical treatment position selection device, and causing the medical treatment position selection device to transmit a medical treatment position according to the corresponding information to the communication terminal.

According to this configuration, the load motion, which is used to specify the medical treatment position, can be clearly instructed to the communication terminal with high reproducibility. Accordingly, the operator (patient or the like) of the communication terminal can accurately execute the load motion with high reproducibility. And then, the communication terminal can transmit, to the medical treatment position selection device, the corresponding information of whether or not the predetermined feeling, such as a pain or the like, occurs in the living body due to the execution of the load motion. The medical treatment position selection device can instruct the operator of the communication terminal of the medical treatment position according to the corresponding information. Accordingly, the communication terminal can display the medical treatment position corresponding to the load motion executed according to the instruction of the medical treatment position selection device with high reproducibility. Therefore, according to the above-described configuration, the curer, such as a doctor or the like, does not need to directly instruct the patient or subject of the load motions, and the people can easily execute the load motions (for specifying the medical treatment positions) by use of the communication terminal, such as a cellular phone or the like. Further, according to the above-described configuration, in a case of training the new curer, the trainer does not need to directly instruct the initiator of the load motions. And then, according to the above-described configuration, the patient himself, who is the operator of the communication terminal, can treat the medical treatment position specified on the basis of the reaction of the living body against the load motion with high reproducibility.

In the medical treatment position selection method according to the third aspect of the invention, it is preferable that the load motion be a movement through the connection of multiple joints and multiple axes of the living body, and the predetermined feeling be a pain.

According to this configuration, when the pain occurs in the living body due to the execution of the load motion, the medical treatment position to be treated on the pain can be displayed in the communication terminal.

In the medical treatment position selection method according to the third aspect of the invention, it is preferable that the medical treatment position be a medical treatment position in oriental medicine, which relates to a system having a trouble in the movement of the living body.

According to this configuration, from therapeutic points, which are used in the acupuncture treatment, the acupressure treatment, and the moxibustion treatment, a therapeutic point to be treated according to the current state of the living body can be accurately instructed with high reproducibility.

In the medical treatment position selection method according to the third aspect of the invention, it is preferable that the load motion be systematized and held in a database connected to Internet.

According to this configuration, for example, with a homepage or the like, the load motions and the medical treatment positions can be simply instructed with high reproducibility.

In the medical treatment position selection method according to the third aspect of the invention, it is preferable that the communication terminal transmit the measurement result on the medical treatment position to the medical treatment position selection device, and the medical treatment position selection device transmit a corresponding processing on the basis of the measurement result to the communication terminal.

According to this configuration, for example, for the living body whose medical treatment position displayed in the communication terminal is treated, a dealing method according to the medical treatment result can be presented. Therefore, it is possible to instruct whether or not a diagnosis by an expert (doctor or the like) needs to be provided with high reproducibility.

According to a fourth aspect of the invention, there is provided a computer program of a medical treatment position selection device, which instructs a communication terminal connected to a network of at least a medical treatment position through the network. The computer program causes a computer to execute transmitting information instructing a load motion as a movement of a living body, to the communication terminal through the network, receiving, from the communication terminal, corresponding information indicating that a predetermined feeling occurs in the living body executing the load motion, and transmitting the medical treatment position according to the corresponding information to the communication terminal through the communication line.

According to this configuration, the load motion can be clearly instructed to the communication terminal with high reproducibility. Accordingly, the operator (patient or the like) of the communication terminal can accurately execute the load motion with high reproducibility. And then, the medical treatment position selection device can receive the corresponding information from the communication terminal, and can instruct the operator of the communication terminal of the medical treatment position according to the corresponding information. The medical treatment position selection device can cause the communication terminal to display the medical treatment position with high reproducibility. Therefore, according to the above-described configuration, the curer, such as a doctor or the like, does not need to directly instruct the patient or subject of the load motions, and the people can easily execute the load motions (for specifying the medical treatment positions) by use of the communication terminal, such as a cellular phone or the like. Further, according to the above-described configuration, in a case of training the new curer, the trainer does not need to directly instruct the initiator of the load motions. And then, according to the above-described configuration, the patient himself, who is the operator of the communication terminal, can treat the medical treatment position specified on the basis of the reaction of the living body against the load motion with high reproducibility.

The computer program according to the fourth aspect of the invention may cause the computer to further execute calculating a fee to be charged on the basis of the number of transmission times or a transmission amount of information instructing the load motion.

According to this configuration, the price according to a use amount of the medical treatment position selection device by the communication terminal can be accurately calculated, and the development of an improved medical treatment position selection device can be promoted by the price.

In the medical treatment position selection system according to the second aspect of the invention, it is preferable that the load motion be executed as a program on the communication terminal, and the medical treatment position selection device or a database store the result of the load motion executed on the communication terminal.

In the medical treatment position selection system according to the second aspect of the invention, it is preferable that the result of the load motion be information indicating that a predetermined feeling occurs in the living body due to the load motion.

According to a fifth aspect of the invention, a medical treatment position selection system includes a communication terminal that is connectable to a communication line, and a medical treatment position selection device having a load motion instructing unit that instructs the communication terminal of a load motion as a movement of a living body, an input unit that inputs the instruction of the load motion, and a medical treatment position instructing unit that instructs a medical treatment position according to corresponding information input by the input unit.

According to a sixth aspect of the invention, a medical treatment position selection method supports a management of a physical state by referring to or transmitting, by electronic mail, the contents of a database, which stores information when a predetermined feeling occurs in a living body due to a load motion while a host selects a medical treatment position.

According to a seventh aspect of the invention, a medical treatment position selection method supports a management of a physical state for a third person assigned by a person who selects a medical treatment position by inputting a plurality of load motion type storage data and load motion degree storage data for plural types of load motions to a load motion type storing unit and a load motion degree storing unit in one unit of measurement, and by referring to or transmitting, by electronic mail, stored contents in at least one unit of measurement.

The medical treatment position selection system according to the second aspect of the invention may further include a load motion type input unit that, when a predetermined feeling occurs in the living body, inputs a type of a specified load motion.

In the medical treatment position selection system according to the second aspect of the invention, it is preferable that, when the predetermined feeling occurs in the living body, the load motion type input unit select and input a degree of the predetermined feeling in the living body according to the type of the specified load motion.

The medical treatment position selection system according to the second aspect of the invention may further include a load motion degree notice unit that, when a predetermined feeling occurs in the living body, digitizes a degree of the predetermined feeling in the living body according to a type of a specified load motion and notices the digitized degree of the predetermined feeling in the living body in advance.

The medical treatment position selection system according to the second aspect of the invention may further include a load motion degree input unit that, when a predetermined feeling occurs in the living body, inputs a degree of the predetermined feeling in the living body according to a type of a specified load motion.

The medical treatment position selection system according to the second aspect of the invention may further include a load motion degree selection and input unit that, when a predetermined feeling occurs in the living body, selects and inputs a degree of the predetermined feeling in the living body according to a type of a specified load motion.

The medical treatment position selection device according to the second aspect of the invention may further include a load motion type storing unit that, when a predetermined feeling occurs in the living body, stores a type of a specified load motion.

The medical treatment position selection system according to the second aspect of the invention may further include a load motion type storage data notice unit that, when the predetermined feeling occurs in the living body, reads out the type of the specified load motion from the load motion type storing unit and notices the type of the specified load motion.

The medical treatment position selection system according to the second aspect of the invention may further include a load motion degree storing unit that, when a predetermined feeling occurs in the living body, stores a degree of the predetermined feeling in the living body according to a type of a specified load motion.

The medical treatment position selection system according to the second aspect of the invention may further include a load motion degree storage data notice unit that, when a predetermined feeling occurs in the living body, reads out a degree of a specified load motion from a load motion degree storing unit and notices the degree of the specified load motion.

In the medical treatment position selection system according to the second aspect of the invention, it is preferable that, when the type of the specified load motion is read out from the load motion type storing unit and is noticed, the degree of the specified load motion be read out from the load motion degree storing unit and be noticed from the load motion degree storage data notice unit in the same unit of notice.

The medical treatment position selection system according to the second aspect of the invention may further include an identification (ID) input unit, with which a person specifies himself to select the medical treatment position. In this case, a plurality of load motion type storage data and load motion degree storage data may be input to the load motion type storing unit and a load motion degree storing unit in one unit of measurement and may be stored therein in at least one unit of measurement.

The medical treatment position selection system according to the second aspect of the invention may further include an ID input unit, with which a person specifies himself to select the medical treatment position, and a medical treatment judging unit that, when a plurality of load motion type storage data and load motion degree storage data for plural types of load motions are set as one unit of measurement, inputs load motion type storage data and load motion degree storage data to the load motion type storing unit and a load motion degree storing unit in different units of measurement, and compares load motion type storage data and load motion degree storage data in different units of measurement.

The medical treatment position selection system according to the second aspect of the invention may further include an ID input unit, with which a person specifies himself to select the medical treatment position. In this case, a plurality of load motion type storage data and load motion degree storage data for plural types of load motions may be set as one unit of measurement. Further, load motion type storage data and load motion degree storage data may be input to the load motion type storing unit and a load motion degree storing unit in different units of measurement, and load motion type storage data and load motion degree storage data may be individually multiplied in different units of measurement.

The medical treatment position selection system according to the second aspect of the invention may further include an ID input unit, with which a person specifies himself to select the medical treatment position. In this case, a plurality of load motion type storage data and load motion degree storage data for plural types of load motions may be set as one unit of measurement. Further, load motion type storage data and load motion degree storage data may be input to the load motion type storing unit and a load motion degree storing unit in different units of measurement, and a sequence of the medical treatment positions associated with the individual load motions may be stored.

The medical treatment position selection device according to the second aspect of the invention may further include an ID input unit, with which a person specifies himself to select the medical treatment position. In this case, a plurality of load motion type storage data and load motion degree storage data for plural types of load motions may be set as one unit of measurement. Further, load motion type storage data and load motion degree storage data may be input to the load motion type storing unit and a load motion degree storing unit in different units of measurement, and a sequence of medical treatment positions associated with the individual load motions may be noticed.

In the medical treatment position selection system according to the second aspect of the invention, it is preferable that the ID input unit, with which the person specifies himself to select the medical treatment position, receive a credit card number and perform an electronic payment for a medical treatment position selection service.

### BRIEF DESCRIPTION OF THE DRAWINGS

The invention will be described with reference to the accompanying drawings, wherein like numbers reference like elements.

FIG. 1 is a block diagram showing a medical treatment position selection device according to a first embodiment of the invention.

FIG. 2A is a diagram showing an example of a load motion.

FIG. 2B is a diagram showing an example of a load motion.

FIG. 2C is a diagram showing an example of a load motion.

FIG. 3A is a diagram showing a meridian distribution in a front body.

FIG. 3B is a diagram showing a meridian distribution in a back body.

FIG. 3A is a diagram showing a meridian distribution in a side body.

FIG. 4 is a diagram showing motion examples of the medical treatment position selection device according to the first embodiment of the invention.

FIG. 5 is a diagram showing a motion picture of load motions, which is displayed by a load motion instructing unit.

FIG. 6 is a block diagram showing a medical treatment position selection system according to a second embodiment of the invention.

FIG. 7 is a flowchart showing an operation of the medical treatment position selection system according to the second embodiment of the invention.

FIG. 8 is a diagram showing a homepage, which is managed by a medical treatment position selection device.

FIG. 9 is a front view of an example of a communication terminal, which constitutes a medical treatment position selection system.

FIG. 10 is a block diagram showing a medical treatment position selection device according to a third embodiment of the invention.

FIG. 11 is a conceptual diagram showing a measuring unit in the medical treatment position selection device.

FIG. 12 is a diagram showing a basic posture of a meridian test and one of load motions.

FIG. 13 is a block diagram of data analysis in a data analyzing device 104.

FIG. 14 is a diagram showing a trace of floor reaction force when a shoulder joint is bent from a basic posture.

### DESCRIPTION OF EXEMPLARY EMBODIMENTS

Hereinafter, a medical treatment position selection device, a medical treatment position selection system, a medical treatment position selection method, and a computer program according to an individual embodiment of the invention will be described with reference to the drawings.

### First Embodiment

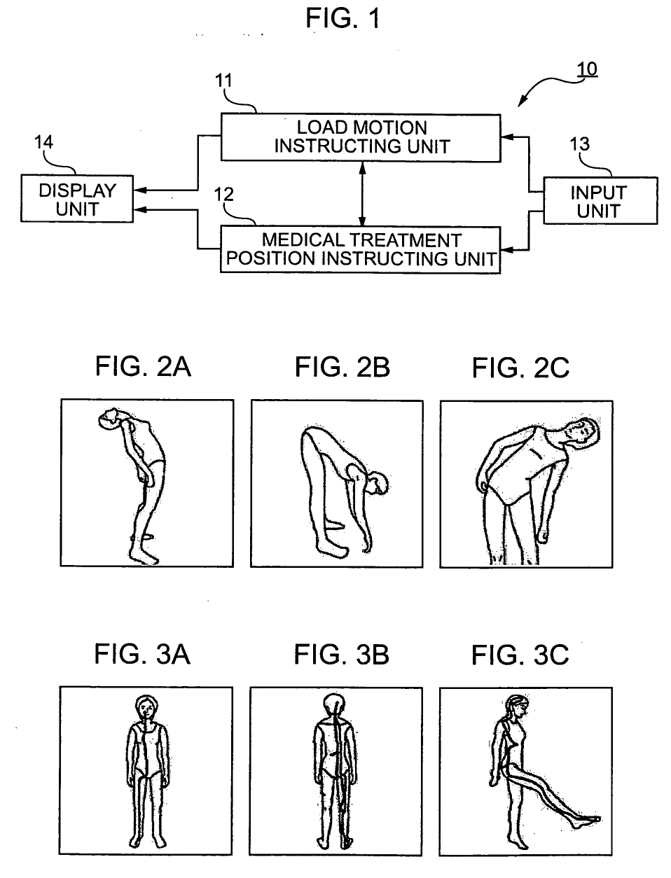

FIG. 1 is a block diagram showing the outline of a medical treatment position selection device 10 according to a first embodiment of the invention. The medical treatment position selection device 10 is a device for causing a person to perform a load motion, which is a movement by the connection of multiple joints and multiple axes in a living body. The medical treatment position selection device 10 has a load motion instructing unit 11, a medical treatment position instructing unit 12, an input unit 13, and a display unit 14.

The load motion instructing unit 11 instructs the load motions. The medical treatment position instructing unit 12 instructs a medical treatment position corresponding to the load motion instructed by the load motion instructing unit 11. The input unit 13 has switches or buttons, which are operated by an operator, such as a patient or a subject, who uses the medical treatment position selection device 10. The display unit 14 displays individual instruction contents of the load motion instructing unit 11 and the medical treatment position instructing unit 12 as motion pictures or still pictures. Moreover, instead of the display unit 14 or together with the display unit 14, a sound generating unit (not shown) may be provided, such that the individual instruction contents of the load motion instructing unit 11 and the medical treatment position instructing unit 12 can be pronounced as sound.

FIGS. 2A to 2C are diagrams illustrating an example of the load motion. The load motions, which are used in the medical treatment position selection device 10, will be described with reference to FIGS. 2A to 2C. FIGS. 2A to 2C show the load motions, in which a torso extends. The load motions of torso extension systematizes the torso of a body into front, back, and side bodies, and specifies regions which limit the connection of muscles, skeleton, and joints in the front, back, or side body. FIG. 2A shows front extension, FIG. 2B shows back extension, and FIG. 2C shows side extension. The person performs the load motions with the living body. On the basis of whether or not the person has a pain through any one of the load motions, the regions, which limit the connection of multiple joints, can be specified.

The meridian is the concept of therapeutic points in the front, back, or side body, and an acupuncture treatment for connecting the therapeutic points. The load motions of torso extension causes the connection of the muscles, skeleton, and multiple joints for the sake of meridian extension.

FIGS. 3A to 3B are diagrams showing a meridian distribution in the front body, the back body, and the side body. The load motions are a system of actions for causing 30 types of pain when any one of meridians of the front body, the back body, or the side body extends. And then, by combining the actions (load motions) for causing 30 types of pain, a region, which is a cause for a pain during a physical movement, is specified.

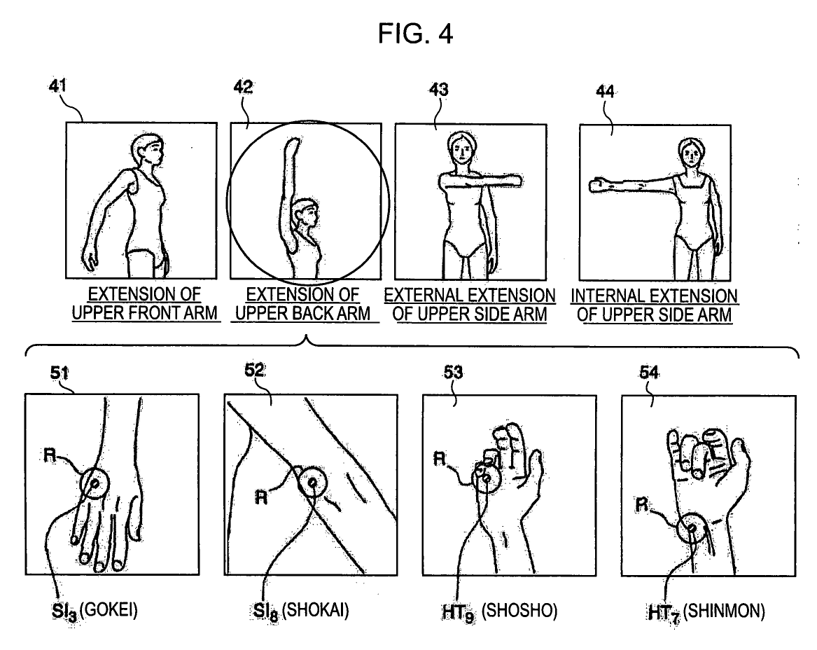

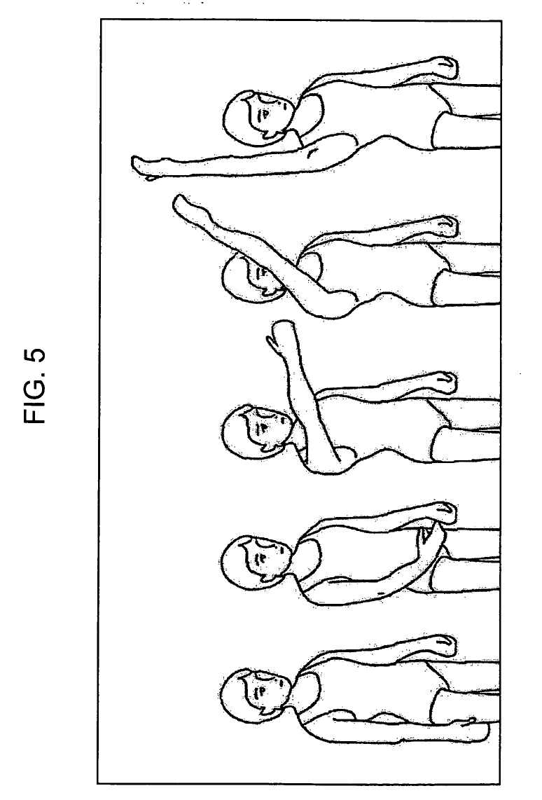

FIG. 4 is a diagram showing an operation example of the medical treatment position selection device 10. In FIG. 4, load motions 41, 42, 43, and 44 instructed by the load motion instructing unit 11, and medical treatment positions 51, 52, 53, and 54 instructed by the medical treatment position instructing unit 12 are shown. The load motions 41, 42, 43, and 44 and the medical treatment positions 51, 52, 53, and 54 are displayed onto the display unit 14 as images on the basis of output signals of the load motion instructing unit 11 or the medical treatment position instructing unit 12.

The load motion 41 shows an extension state of an upper front arm. The load motion 42 shows an extension state of an upper back arm. The load motion 43 shows an external extension state of an upper side arm. The load motion 44 shows an internal extension state of the upper side arm. As such, the load motion instructing unit 11 instructs plural types of load motions. The operator can select one of a plurality of load motions 41, 42, 43, and 44 by use of the input unit 13.

The medical treatment positions 51, 52, 53, and 54 show the medical treatment positions associated with the load motion 42 from the plurality of load motions 41, 42, 43, and 44. That is, when a pain occurs by the extension motion of the upper back arm as the load motion 42, the medical treatment positions 51, 52, 53, and 54 shows therapeutic points for medical treatment (therapeutic points in the meridian of the back body). The medical treatment position 51 is GOKEI SI₃. The medical treatment position 52 is SHOKAI SI₈. The medical treatment position 53 is SHOSHO HT₉. The medical treatment position 54 is SHINMON HT₇. When the extension motion of the upper back arm is repeated while the medical treatment positions 51, 52, 53, and 54 serving as four therapeutic points are sequentially pressed, the occurrence of a pain of the upper back arm can be caught.

And then, the medical treatment position instructing unit 12 preferably has a medical treatment method presenting unit (not shown) that causes the display unit 14 to display a sentence purporting that ‘when the extension motion of the upper back arm is repeated while the medical treatment positions 51, 52, 53, and 54 are sequentially pressed, the pain of the upper back arm can be caught’.

Further, as for other load motions 41, 43, and 44, like the load motion 42, a plurality of medical treatment positions are associated. Therefore, the medical treatment position instructing unit 12 can instruct the medical treatment positions associated with the individual load motions. Further, as shown in FIG. 4, the medical treatment position instructing unit 12 causes the display unit 14 to display a ring R surrounding the therapeutic points of the individual medical treatment positions 51, 52, 53, and 54. Accordingly, the individual medical treatment positions 51, 52, 53, and 54 can be displayed clearly and easily to understand.

FIG. 5 is a diagram showing the load motion 41 as a motion picture. The motion picture is sequentially played from a state on the left side of FIG. 5 to a state on the right side thereof. The motion picture shown in FIG. 5 is also a load motion instructed by the load motion instructing unit 11 to be displayed onto the display unit 14. The motion picture shown in FIG. 5 is preferably a three-dimensional computer graphic. Further, the load motions 41, 42, 43, and 44 and the medical treatment positions 51, 52, 53, and 54 shown in FIG. 4 are preferably three-dimensional computer graphics. Moreover, the motions or the positions shown in FIG. 4 or 5 may be specified by use of sound or the like.

Next, the operation example of the medical treatment position selection device 10 will be described. For example, the operator, such as a patient or the like, operates the input unit 13 so as to click and specify the region of the load motion 42 in FIG. 4. If doing so, the load motion instructing unit 11 causes the display unit 14 to display the motion picture indicating the load motion 42 in FIG. 5. On the basis of the instruction by image display, the operator performs the load motion 42. And then, when the operator feels a pain by performing the load motion 42, the operator clicks the region of the load motion 42 in FIG. 4. If doing so, the medical treatment position instructing unit 12 causes the display unit 14 to display the medical treatment positions 51, 52, 53, and 54 shown in FIG. 4. The operator performs acupuncture, moxibustion, acupressure, or massage according to the instruction of the medical treatment position instructing unit 12 on the medical treatment positions 51, 52, 53, and 54 displayed onto the display unit 14.

As such, by using the medical treatment position selection device 10, the operator can effectively treat his body by himself. Further, the operator can learn a medical treatment position method, such as acupuncture, moxibustion, acupressure, or massage, with high reproducibility. Therefore, according to the medical treatment position selection device 10, reproducibility of the load motion instruction of the same person due to the personal tradition of the load motions and reproducibility of the load motion instruction among many curers can be ensured. Further, in a case of training a new curer, a trainer does not need to directly instruct an initiator of the load motions.

Next, an application of the medical treatment position selection device 10 of the present embodiment will be described.

In the above-described embodiment, the acupuncture treatment in oriental medicine has been described, but the invention is not limited thereto. The load motions can be widely applied to a physical treatment in western medicine and a specified function recovery treatment in rehabilitation.

Further, the medical treatment position selection device 10 may be a small and portable apparatus, such as a cellular phone or the like. Accordingly, diagnosis and treatment in home can be performed. Further, the medical treatment position selection device 10 can be under medical institute control and also can be applied to a self-care field.

In addition, like a second embodiment described below, the medical treatment position selection device 10 can have a communication function. Accordingly, various kinds of input information can be input to the medical treatment position selection device 10 through a communication line and various kinds of output information can be transmitted from the medical treatment position selection device 10 to the outside through the communication line.

### Second Embodiment

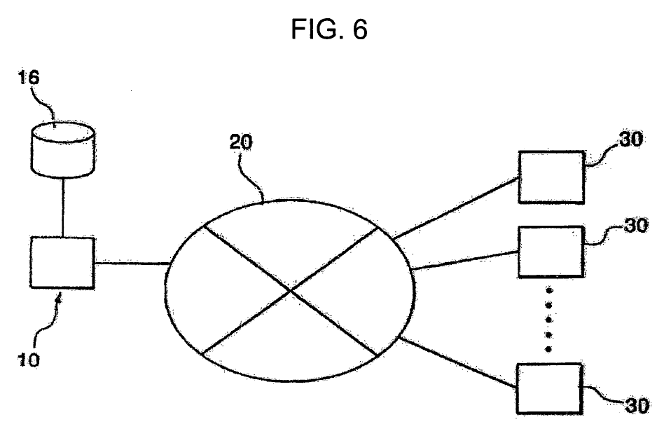

FIG. 6 is a block diagram showing a medical treatment position selection system according to the second embodiment of the invention.

The medical treatment position selection system has the medical treatment position selection device 10 according to the first embodiment of the invention, a load motion database 16, an electrical communication line 20, and a communication terminal 30.

The medical treatment position selection device 10 has a function as a WEB (World Wide Web) server, in addition to the functions described in the first embodiment of the invention. That is, the medical treatment position selection device 10 has a communication function of receiving an information presentation request from the communication terminal 30 through the communication line 20 and providing information corresponding to the information presentation request to the communication terminal 30. With such a communication function, the load motion instructing unit 11 instructs the communication terminal 30 of the load motions as the movements in the living body through the communication line 20. The input unit 13 receives the corresponding information from the communication terminal 30, which receives the load motion instruction with the communication function. Further, the medical treatment position instructing unit 12 instructs the communication terminal 30 of the medical treatment positions according to the corresponding information, which is received by the input unit with the communication function, through the communication line 20.

The communication line 20 is not limited to a telephone network, but includes Internet. The load motion database 16 systematizes and stores the load motions described in the first embodiment of the invention, and has a function corresponding to the WEB. The load motion database 16 can provide the load motions according to the request of the medical treatment position selection device 10 to other devices (the medical treatment position selection device 10 or the communication terminal 30). The communication terminal 30 is a device, which perform a communication through the communication line 20. For example, a personal computer, a PDA (Personal Digital Assistant), or a cellular phone can be used. Further, the communication terminal 30 has, a function as a WEB browser, which can communicate with at least the medical treatment position selection device 10.

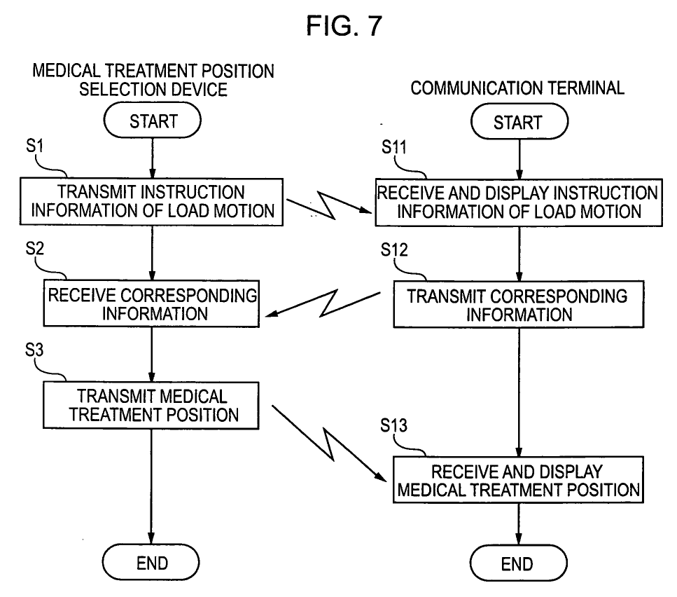

Next, FIG. 7 is a flowchart showing the summary operation of the medical treatment position selection system according to the present embodiment. First, the load motion instructing unit 11 of the medical treatment position selection device 10 transmits information for instructing the load motion to the communication terminal 30 through the communication line (Step S1). Here, the load motion instructing unit 11 accesses to the load motion database 16, selects a desired load motion from the multiple load motions held in the load motion database 16, and transmits the selected load motion to the communication terminal 30.

When the load motion is received, the communication terminal 30 displays the instructed load motion onto a display unit of the communication terminal 30 (Step S11). Here, the communication terminal 30 displays the instructed load motion by use of a motion picture or a still picture.

Next, the operator (a patient or the like) performs the load motion displayed onto the communication terminal 30. If a predetermined feeling, such as a pain or the like, occurs by the load motion, the operator inputs information indicating the occurrence to the communication terminal 30. When the input at the time of the occurrence of the predetermined feeling is received, the communication terminal 30 transmits, to the medical treatment position selection device 10, corresponding information indicating that the predetermined feeling occurs (Step S12).

Next, the medical treatment position selection device 10 receives the corresponding information transmitted from the communication terminal 30 (Step S2). Subsequently, the medical treatment position selection device 10 transmits the medical treatment position according to the received corresponding information to the communication terminal 30 through the communication line 20 (Step S3).

Next, the communication terminal 30 receives the medical treatment position and displays the medical treatment position onto the display unit of the communication terminal 30 (Step S13).

Accordingly, the operator of the communication terminal 30 can treat the medical treatment position displayed onto the display unit of the communication terminal 30 personally. Here, the medical treatment position is preferably a medical treatment position in oriental medicine, which relates to a system having a trouble in the movement in the living body. That is, the medical treatment position is preferably a therapeutic point, which is used for an acupuncture treatment, an acupressure treatment, a moxibustion treatment, or the like. Therefore, as the medical treatment, which is personally executed by the operator, the acupuncture treatment, the acupressure treatment, the moxibustion treatment, or the like can be exemplified. Further, the communication terminal 30 preferably displays the medical treatment position by using the motion picture or the still picture. Moreover, the communication terminal 30 may represent the load motion or the medical treatment position with sound.

Therefore, according to the medical treatment position selection system of the present embodiment, the curer, such as a doctor or the like, does not need to directly instruct the patient or subject of the load motions. Further, by use of the communication terminal 30, such as a cellular phone or the like, the people can easily and accurately perform the load motions for specifying the medical treatment positions. In addition, according to the medical treatment position selection system, the trainer does not need to directly instruct the initiator to the load motions.

Further, The medical treatment position selection device 10 may have a step of calculating a fee to be charged on the basis of the number of execution times of the step S1 and the step S3 or the amount of transmitted information in the step S1 and the step S3. If doing so, a price according to a use amount of the medical treatment position selection device 10 by the communication terminal 30 can be accurately calculated.

Further, the medical treatment position selection device 10 may receive or store the medical treatment result of the instructed medical treatment position from the communication terminal 30, and may transmit a corresponding processing on the basis of the medical treatment result to the communication terminal. If doing so, for example, as for the living body whose medical treatment position displayed onto the communication terminal 30 is treated, a dealing measure according to the medical treatment result can be presented. Therefore, whether or not a diagnosis by an expert (doctor or the like) needs to be provided can be presented with high reproducibility. Next, a specified example of the medical treatment position selection system of the present embodiment will be described.

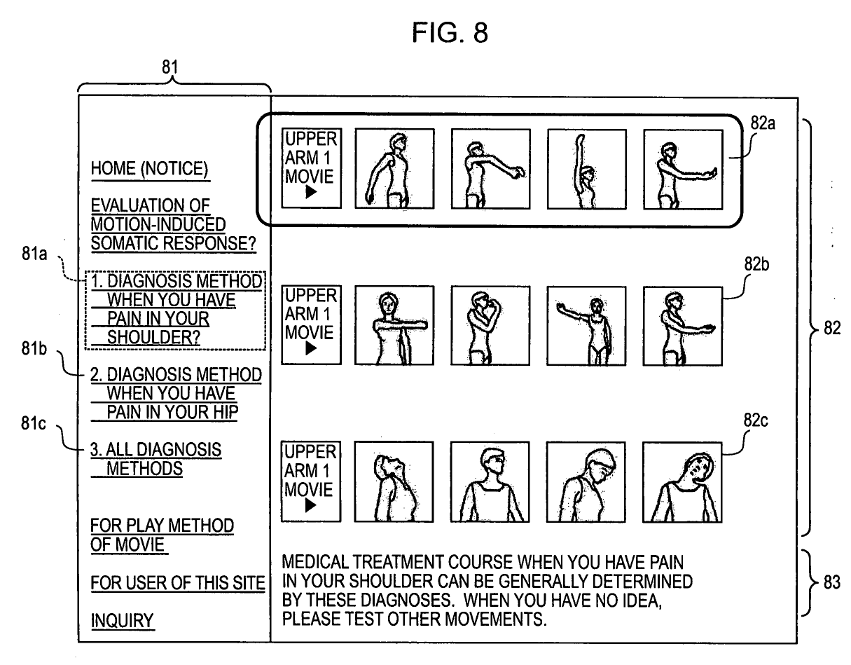

FIG. 8 is a diagram showing an example of a homepage, which is managed by the medical treatment position selection device 10. The medical treatment position selection device 10 has a function as a server for managing the load motion database 16 and the homepage shown in FIG. 8. And then, the communication terminal 30 has a function as a client for using the homepage managed by the medical treatment position selection device 10. Therefore, the medical treatment position selection device 10 causes the display unit of the communication terminal 30 to display the homepage shown in FIG. 8 according to the request of the communication terminal 30 through the communication line 20.

The homepage shown in FIG. 8 has a contents region 81, an image display region 82, and a medical treatment explanation region 83. The contents region 81 has ‘indexes’ of a plurality of diagnosis methods, that is, diagnosis methods 81a, 81b, and 81c. The diagnosis method 81a is a diagnosis method when a user has a pain in his shoulder. The diagnosis method 81b is a diagnosis method when the user has a pain in his hip. The diagnosis method 81c relates to all diagnosis methods. The image display region 82 is a region in which the load motions corresponding to any one of the diagnosis methods 81a, 81b, and 81c are displayed as images. And then, in the image display region 82, the load motions on one diagnosis method specified by the operation of the communication terminal 30 from the diagnosis methods 81a, 81b, and 81c are displayed. In the medical treatment explanation region 83, the explanation about the load motions (or diagnosis method) displayed in the image display region 82 is displayed.

Next, the operation example of the medical treatment position selection system will be described. For example, the operator (patient or the like) accesses the homepage shown in FIG. 8 through the operation of the communication terminal 30, and clicks the diagnosis method 81a, which is the diagnosis method when the user has the pain in his shoulder in FIG. 8. If doing so, in the image display region 82, a first load motion 82a, a second load motion 82b, and a third load motion 82c, which are the load motions corresponding to the diagnosis method 81a, are displayed.

In addition, if the operator clicks the first load motion 82a by operating the communication terminal 30, the motion picture indicating the load motions shown in FIG. 5 is displayed onto the communication terminal 30. Such a motion picture is displayed for each of the first load motion 82a, the second load motion 82b, and the third load motion 82c. On the basis of the instruction by display, the operator of the communication terminal 30 executes the individual load motions.

Further, like the first load motion 82a, the second load motion 82b, and the third load motion 82c, the load motions 41, 42, 43, and 44 shown in FIG. 4 can be displayed onto the communication terminal 30 as the motion pictures, and thus the operator can execute the load motions. That is, FIG. 4 is another part (or link destination) of the homepage, which is managed by the medical treatment position selection device 10. The operator can click and specify any one of the plurality of load motions 41, 42, 43, and 44 by use of the communication terminal 30. And then, the specified load motion is displayed onto the display unit of the communication terminal 30 as the motion picture, as shown in FIG. 5.

In the execution of each load motion, if the operator feels a pain by the load motion 42, for example, the operator clicks the region of the load motion 42 in FIG. 4. If doing so, as shown in FIG. 4, the medical treatment positions 51, 52, 53, and 54 linked to the load motion 42 are displayed onto the communication terminal 30. The operator performs acupuncture, moxibustion, acupressure, or massage on the displayed medical treatment positions 51, 52, 53, and 54 according to the instruction of the homepage (the medical treatment position instructing unit 12).

Accordingly, the operator of the communication terminal 30 can effectively treat his body personally. Further, the operator of the communication terminal 30 can learn the medical treatment method of acupuncture, moxibustion, acupressure, or massage with high reproducibility. Therefore, according to the medical treatment position selection system of the present embodiment, reproducibility of the load motion instruction of the same person due to the personal tradition of the load motions and reproducibility of the load motion instruction among many curers can be ensured. Further, in a case of training the new curer, the trainer does not need to directly instruct the initiator of the load motions.

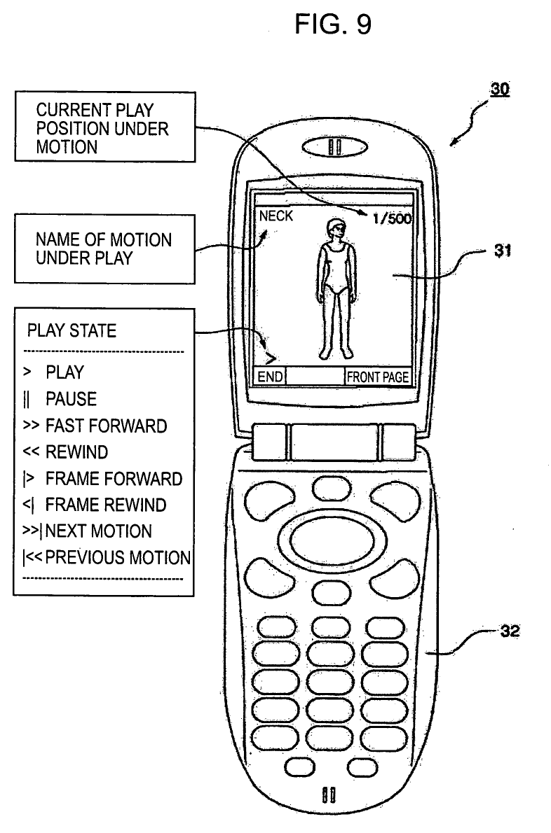

FIG. 9 is a front view showing an example of the communication terminal 30. The communication terminal 30 is a cellular phone. The communication terminal 30 has a display unit 31 and an operating unit 32. The operator operates the operating unit 32 so as to access to the homepage shown in FIG. 8. In addition, the operator operates the operating unit 32 so as to cause the display unit 31 to display the load motions linked to the homepage, as shown in FIGS. 8 and 5.

Further, the communication terminal 30 itself may constitute the medical treatment position selection device 10. That is, the medical treatment position selection device 10 may be provided as one function of the cellular phone. In this case, various load motions may be integrated with a database and may be stored in a storing unit of the cellular phone. Further, the communication terminal 30 displays information indicating a current play position of a motion as the load motion in a partial region of the display unit 31. Further, the communication terminal 30 displays a motion name (load motion name) under play in a partial region of the display unit 31. In addition, the communication terminal 30 displays a symbol indicating a play state of the load motion. The play state includes play, pause, fast forward, rewind, frame forward, frame rewind, next motion, and previous motion.

### Third Embodiment

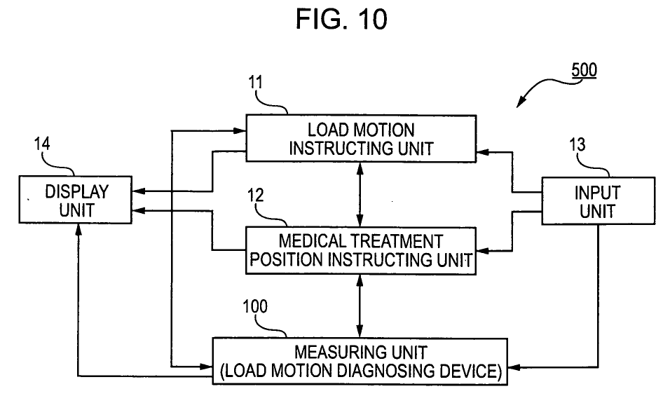

FIG. 10 is a block diagram showing a medical treatment position selection device 500 according to a third embodiment of the invention. Moreover, in FIG. 10, the same parts as those in the medical treatment position selection device 10 shown in FIG. 1 are represented by the same reference numerals. The medical treatment position selection device 500 of the present embodiment has a load motion instructing unit 11, a medical treatment position instructing unit 12, an input unit 13, a display unit 14, and a measuring unit (load motion diagnosing device) 100. Therefore, the medical treatment position selection device 500 has a configuration in which the measuring unit 100 is added to the medical treatment position selection device 10 of the first embodiment of the invention.

The measuring unit 100 performs the measurement of the living body whose medical treatment positions instructed by the medical treatment position instructing unit 12 is stimulated or treated. For example, the operator performs various load motions on the basis of the instruction of the load motion instructing unit 11. Here, when a pain occurs by one load motion, the operator operates the input unit 13 so as to specify the load motion which causes the pain. If doing so, the medical treatment position instructing unit 12 instructs the medical treatment position corresponding to the specified load motion. On the basis of the instruction, the operator performs the acupressure or the like on the medical treatment position. And then, the measuring unit 100 measures the living body so as to judge whether or not the acupressure or the like is effective.

Accordingly, the medical treatment position selection device 500 can instruct the load motion with high reproducibility, can accurately select the medical treatment position on the basis of the load motion, and can quantitatively confirm the effect, such as the stimulation of the medical treatment position or the like.

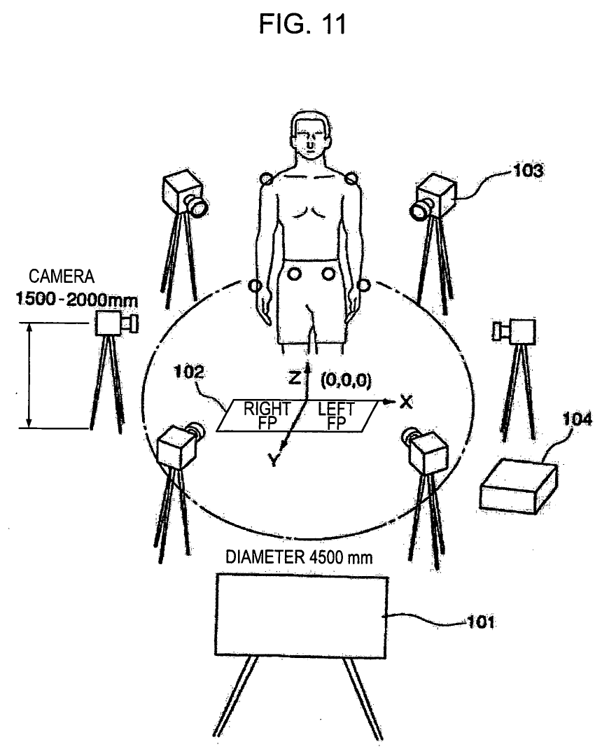

FIG. 11 is a conceptual diagram showing an example of the measuring unit 100. The measuring unit 100 constitutes the load motion diagnosing device that finds the limitation to the movement of the patient and the subject (operator) and confirms the medical treatment by the load motions as the movements through the connection of multiple joints and multiple axes of the living body. That is, the measuring unit 100 constitutes a medical treatment result judging unit that, on the basis of the medical treatment result, judges whether or not an abnormality is reduced in the living body whose medical treatment position instructed by the medical treatment position instructing unit 12 is stimulated (or treated).

A load motion instructing unit 101 is a load motion instructing unit that instructs a basic posture of the meridian test and a motion of bending a shoulder joint, which is one of the load motions, that is, a motion of raising a lowered arm while an elbow extends. Therefore, the load motion instructing unit 101 can be substituted with the load motion instructing unit 11 of the medical treatment position selection device 500. To the contrary, the load motion instructing unit 11 of the medical treatment position selection device 500 can be substituted with the load motion instructing unit 101. The load motion instructing unit 101 ends the load motions in four seconds from the basis posture according to metronome sound of 60 beats/minute for defining the load motion.

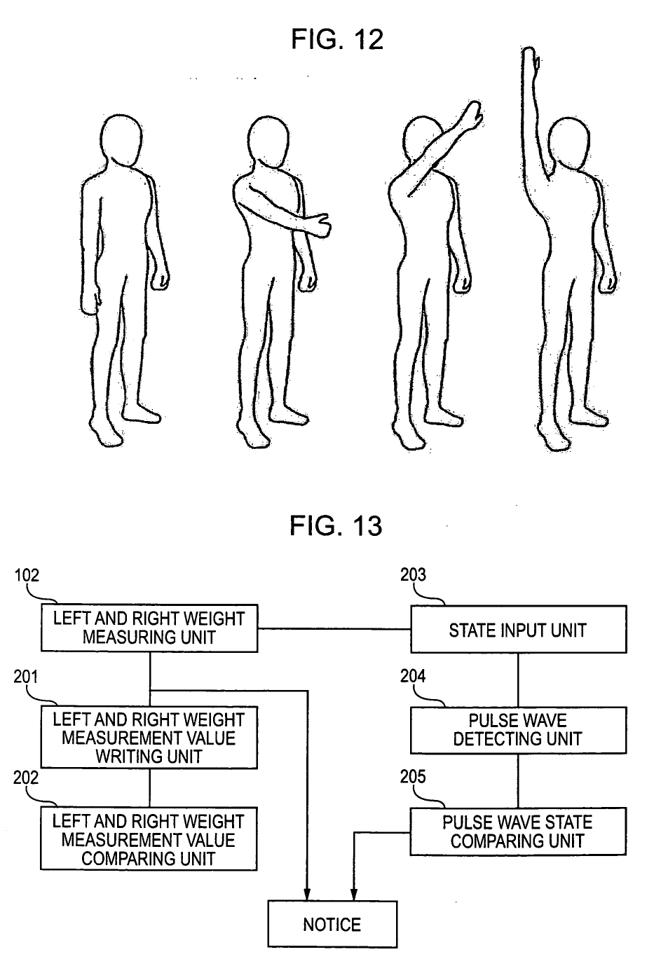

FIG. 12 is a diagram showing the motion of raising the lowered arm while the elbow extends. A left and right weight measuring unit 102 measures weights of left and right legs by two floor reaction force meter on the left and right sides. For example, six infrared cameras 13 are arranged at constant intervals at the height of 1500 to 2000 mm on a circumference having a diameter of 4500 mm, and measurement is performed for each motion in synchronization with an analog signal of the floor reaction force meter at a sampling frequency of 120 Hz. The data analyzing device 104 is a device for analyzing data of the left and right weight measuring unit 102.

FIG. 13 is a block diagram of a data analysis in the data analyzing device 104. If the subject performs the load motion by the instruction of the load motion instructing unit 101, data measured by the left and right weight measuring unit 102 is written into the left and right weight measurement value writing unit 201, and the measuring values are notices. Further, measured data, that is, the left and right weight measurement values are compared with each other in a left and right weight measurement value comparing unit 202.

A state input unit 203 inputs a stimulation state on the living body, and the measurement of the left and right weights are performed at least two times to correspond to the stimulation state. A pulse wave detecting unit 204 noninvasively detects pulse waves in a periphery at least two times to correspond to the stimulation state of the state input unit 203. Further, with the state input unit 203, the stimulation state is input, and the pulse waves corresponding to at least two stimulation states are detected. A pulse wave state comparing unit 205 compares the detection value of the pulse wave corresponding to at least two stimulation states. And then, the detection value of the pulse wave corresponding to at least two stimulation states is compared by the pulse wave state comparing unit 205. When the detection value exceeds a predetermined range, it is noticed that a difference in pulse wave exists between at least two stimulation states.

Here, as a specified example, an experiment, in which the measuring unit (load motion diagnosing device) 100 is used, will be described below in detail. The subjects are 20 men having a ping-pong career of 7 years or more. Dominant hands and legs of the subjects are shown in Table 1. Markers having a diameter of 9 mm are set to both wrists, both shoulders, and a pelvis of each subject. Further, in Table 1, in addition to the dominant hands and legs of the subjects, the positions of the markers of the shoulder and hip of the Z axis in the basic posture before acupuncture stimulation are shown. 14 men of M22C, M19D, M18E, M21G, M19H, M21I, M21J, M18K, M18L, M20M, M20O, M21P, M21Q, and M21R exhibit a significant difference in position of the markers of the left and right-shoulders. On the other hand, 13 men of M18A, M18B, M22C, M19D, M20F, M21G, M21I, M18K, M20M, M20O, M21P, M21Q, and M18T exhibit a significant difference in position of the markers of the left and right hips. Nine men have lateral differences in the shoulder and the hip, five examples have the lateral difference only in the shoulder, and three examples have the lateral difference only in the hip. Only two examples do not exhibit the lateral difference in the shoulder and the hip. Almost all subjects have a slant in the basic posture.

TABLE 1
Dominant hands and legs of subjects and positions of markers in Z axis before
stimulation (mm)

| | | | MARKER OF | | | MARKER OF | | |
|---|---|---|---|---|---|---|---|---|
| | | | SHOULDER | | | PELVIS | | |
| | DOMINANT | DOMINANT | LEFT | RIGHT | TEST | LEFT | RIGHT | TEST |
| SUBJECT | HAND | LEG | SHOULDER | SHOULDER | RESULT | HIP | HIP | RESULT |
| M18A | RIGHT | RIGHT | 1394 ± 3 | 1391 ± 1 | — | 945 ± 0 | 950 ± 1 | ** |
| M18B | RIGHT | RIGHT | 1328 ± 2 | 1322 ± 2 | — | 927 ± 1 | 923 ± 1 | * |
| M22C | RIGHT | RIGHT | 1302 ± 3 | 1314 ± 3 | ** | 870 ± 0 | 860 ± 0 | *** |
| M19D | LEFT | RIGHT | 1257 ± 2 | 1261 ± 2 | * | 860 ± 0 | 836 ± 0 | *** |
| M18E | RIGHT | LEFT | 1429 ± 1 | 1423 ± 1 | * | 976 ± 0 | 977 ± 0 | — |
| M20F | RIGHT | RIGHT | 1358 ± 3 | 1345 ± 4 | — | 910 ± 1 | 914 ± 1 | * |
| M21G | RIGHT | RIGHT | 1390 ± 1 | 1375 ± 3 | * | 892 ± 0 | 881 ± 2 | ** |
| M19H | RIGHT | RIGHT | 1368 ± 2 | 1357 ± 2 | ** | 926 ± 0 | 926 ± 1 | — |
| M21I | RIGHT | LEFT | 1344 ± 2 | 1330 ± 1 | ** | 908 ± 3 | 915 ± 3 | * |
| M21J | RIGHT | RIGHT | 1501 ± 3 | 1473 ± 2 | ** | 1009 ± 1 | 1007 ± 1 | — |
| M18K | RIGHT | LEFT | 1378 ± 2 | 1364 ± 4 | * | 926 ± 2 | 930 ± 1 | * |
| M18L | RIGHT | RIGHT | 1363 ± 1 | 1344 ± 2 | * | 913 ± 1 | 915 ± 1 | — |
| M20M | RIGHT | LEFT | 1385 ± 2 | 1361 ± 2 | *** | 925 ± 1 | 919 ± 1 | ** |
| M20N | LEFT | RIGHT | 1335 ± 4 | 1340 ± 1 | — | 908 ± 1 | 907 ± 1 | — |
| M20O | RIGHT | RIGHT | 1404 ± 1 | 1367 ± 2 | * | 943 ± 0 | 947 ± 1 | * |
| M21P | RIGHT | LEFT | 1378 ± 3 | 1369 ± 2 | * | 923 ± 1 | 921 ± 1 | * |
| M20Q | RIGHT | LEFT | 1429 ± 6 | 1411 ± 0 | * | 949 ± 3 | 964 ± 2 | *** |
| M21R | RIGHT | RIGHT | 1355 ± 1 | 1325 ± 1 | *** | 903 ± 2 | 902 ± 2 | — |
| M18S | RIGHT | RIGHT | 1431 ± 0 | 1436 ± 6 | — | 959 ± 1 | 962 ± 1 | — |
| M18T | RIGHT | LEFT | 1376 ± 4 | 1375 ± 4 | — | 898 ± 2 | 890 ± 3 | ** |

* P < 0.05
** P < 0.01
*** P < 0.001

Table 2 shows lateral differences of markers of a shoulder and a hip in the basic posture after acupuncture stimulation. In four examples of M18B, M19D, M20F, and M21P, the lateral difference of the marker of the shoulder is newly made significant or a significant lateral difference disappears, that is, the lateral difference of the marker of the shoulder is changed. In five examples of M22C, M18E, M18K, M18L, and M21P, the lateral difference of the marker of the hip is newly made significant or a significant lateral difference disappears, that is, the lateral difference of the marker of the hip is changed. M21P has a change in both the shoulder and the hip. Eight examples have a change in basic posture after the acupuncture stimulation. It was supposed that the acupuncture stimulation caused the change in posture.

TABLE 2
Marker positions in Z axis after stimulation of dominant hands and legs of
subjects (mm)

| | | | MARKER OF | | | MARKER OF | | |
|---|---|---|---|---|---|---|---|---|
| | | | SHOULDER | | | PELVIS | | |
| | DOMINANT | DOMINANT | LEFT | RIGHT | TEST | LEFT | RIGHT | TEST |
| SUBJECT | HAND | LEG | SHOULDER | SHOULDER | RESULT | HIP | HIP | RESULT |
| M18A | RIGHT | RIGHT | 1395 ± 3 | 1391 ± 1 | — | 944 ± 0 | 949 ± 1 | * |
| M18B | RIGHT | RIGHT | 1328 ± 1 | 1321 ± 1 | * | 925 ± 1 | 921 ± 1 | * |
| M22C | RIGHT | RIGHT | 1302 ± 2 | 1311 ± 0 | * | 869 ± 1 | 861 ± 6 | — |
| M19D | LEFT | RIGHT | 1258 ± 2 | 1259 ± 4 | — | 860 ± 1 | 836 ± 2 | ** |
| M18E | RIGHT | LEFT | 1429 ± 1 | 1424 ± 1 | * | 975 ± 1 | 977 ± 1 | ** |
| M20F | RIGHT | RIGHT | 1357 ± 3 | 1343 ± 0 | * | 909 ± 0 | 914 ± 0 | *** |
| M21G | RIGHT | RIGHT | 1388 ± 2 | 1373 ± 1 | * | 894 ± 1 | 882 ± 0 | ** |
| M19H | RIGHT | RIGHT | 1371 ± 3 | 1360 ± 2 | ** | 924 ± 1 | 925 ± 1 | — |
| M21I | RIGHT | LEFT | 1343 ± 1 | 1327 ± 1 | *** | 907 ± 2 | 918 ± 2 | *** |
| M21J | RIGHT | RIGHT | 1497 ± 3 | 1471 ± 2 | ** | 1006 ± 1 | 1006 ± 2 | — |
| M18K | RIGHT | LEFT | 1373 ± 5 | 1359 ± 3 | * | 924 ± 1 | 929 ± 1 | — |
| M18L | RIGHT | RIGHT | 1364 ± 4 | 1342 ± 3 | ** | 913 ± 1 | 914 ± 2 | * |
| M20M | RIGHT | LEFT | 1383 ± 2 | 1358 ± 3 | ** | 925 ± 1 | 920 ± 1 | * |
| M20N | LEFT | RIGHT | 1336 ± 3 | 1341 ± 3 | — | 906 ± 2 | 906 ± 1 | — |
| M20O | RIGHT | RIGHT | 1411 ± 2 | 1372 ± 1 | *** | 942 ± 1 | 947 ± 0 | ** |
| M21P | RIGHT | LEFT | 1381 ± 1 | 1375 ± 6 | — | 923 ± 2 | 922 ± 3 | — |
| M20Q | RIGHT | LEFT | 1425 ± 2 | 1412 ± 2 | * | 949 ± 2 | 964 ± 2 | ** |
| M21R | RIGHT | RIGHT | 1355 ± 0 | 1324 ± 1 | *** | 903 ± 1 | 902 ± 1 | — |
| M18S | RIGHT | RIGHT | 1431 ± 4 | 1435 ± 1 | — | 959 ± 2 | 960 ± 1 | — |
| M18T | RIGHT | LEFT | 1377 ± 13 | 1370 ± 13 | — | 899 ± 1 | 889 ± 1 | * |

* P < 0.05
** P < 0.01
*** P < 0.001

Table 3 shows lateral differences of floor reaction force in the basic posture before stimulation. In M22C, M19D, M21J, and M18T, a significant difference was viewed. Table 4 shows lateral differences of floor reaction force in the basic posture after stimulation. In M22C, M19D, M21J, and M18T, the significant difference, which was observed before stimulation, disappeared. After stimulation, in M200, M21P, and M21R, a significant lateral difference newly occurred. In seven examples in total, the change of the lateral difference in the basic posture after acupuncture stimulation occurred. It was supposed that the acupuncture stimulation caused the change in center movement. From eight examples having the change in the basic posture after the acupuncture stimulation, only three examples of M22C, M19D, and M21P have also the change in the lateral difference of the floor reaction force. Four examples of M21J, M18T, M200, and M21R have a significant change in the lateral difference of the floor reaction force meter, regardless of not having a significant lateral difference of the marker of the shoulder or hip.

TABLE 3
Lateral difference of floor reaction force before stimulation
of dominant hands and legs of subjects (N)

| | | | FLOOR REACTION FORCE | | |
|---|---|---|---|---|---|
| | DOM- | DOM- | (BEFORE STIMULATION) | | |
| | INANT | INANT | | | TEST |
| SUBJECT | HAND | LEG | LEFT FP | RIGHT FP | RESULT |
| M18A | RIGHT | RIGHT | 261 ± 7 | 253 ± 6 | — |
| M18B | RIGHT | RIGHT | 263 ± 5 | 252 ± 4 | — |
| M22C | RIGHT | RIGHT | 278 ± 2 | 304 ± 4 | * |
| M19D | LEFT | RIGHT | 251 ± 1 | 222 ± 5 | * |
| M18E | RIGHT | LEFT | 308 ± 13 | 325 ± 14 | — |
| M20F | RIGHT | RIGHT | 280 ± 19 | 292 ± 15 | — |
| M21G | RIGHT | RIGHT | 304 ± 15 | 293 ± 15 | — |
| M19H | RIGHT | RIGHT | 299 ± 6 | 301 ± 7 | — |
| M21I | RIGHT | LEFT | 346 ± 4 | 325 ± 5 | — |
| M21J | RIGHT | RIGHT | 331 ± 4 | 308 ± 3 | * |
| M18K | RIGHT | LEFT | 311 ± 4 | 316 ± 8 | — |
| M18L | RIGHT | RIGHT | 317 ± 7 | 274 ± 12 | — |
| M20M | RIGHT | LEFT | 312 ± 15 | 305 ± 11 | — |
| M20N | LEFT | RIGHT | 310 ± 16 | 279 ± 15 | — |
| M20O | RIGHT | RIGHT | 295 ± 11 | 255 ± 10 | — |
| M21P | RIGHT | LEFT | 384 ± 11 | 299 ± 16 | — |
| M20Q | RIGHT | LEFT | 271 ± 7 | 290 ± 12 | — |
| M21R | RIGHT | RIGHT | 233 ± 11 | 285 ± 14 | — |
| M18S | RIGHT | RIGHT | 287 ± 21 | 280 ± 25 | — |
| M18T | RIGHT | LEFT | 302 ± 6 | 341 ± 6 | * |

* P < 0.05

TABLE 4
Lateral difference of floor reaction force after stimulation
of dominant hands and legs of subjects (N)

| | | | FLOOR REACTION FORCE | | |
|---|---|---|---|---|---|
| | DOM- | DOM- | (AFTER STIMULATION) | | |
| | INANT | INANT | | | TEST |
| SUBJECT | HAND | LEG | LEFT FP | RIGHT FP | RESULT |
| M18A | RIGHT | RIGHT | 245 ± 14 | 245 ± 14 | — |
| M18B | RIGHT | RIGHT | 271 ± 16 | 243 ± 13 | — |
| M22C | RIGHT | RIGHT | 277 ± 8 | 311 ± 9 | — |
| M19D | LEFT | RIGHT | 256 ± 10 | 226 ± 11 | — |
| M18E | RIGHT | LEFT | 319 ± 4 | 324 ± 3 | — |
| M20F | RIGHT | RIGHT | 282 ± 7 | 301 ± 4 | — |
| M21G | RIGHT | RIGHT | 302 ± 6 | 296 ± 5 | — |
| M19H | RIGHT | RIGHT | 295 ± 12 | 303 ± 13 | — |
| M21I | RIGHT | LEFT | 359 ± 25 | 325 ± 22 | — |
| M21J | RIGHT | RIGHT | 332 ± 9 | 309 ± 5 | — |
| M18K | RIGHT | LEFT | 309 ± 7 | 315 ± 12 | — |
| M18L | RIGHT | RIGHT | 302 ± 2 | 296 ± 15 | — |
| M20M | RIGHT | LEFT | 320 ± 12 | 300 ± 19 | — |
| M20N | LEFT | RIGHT | 299 ± 4 | 290 ± 4 | — |
| M20O | RIGHT | RIGHT | 305 ± 3 | 248 ± 2 | ** |
| M21P | RIGHT | LEFT | 351 ± 5 | 317 ± 8 | * |
| M20Q | RIGHT | LEFT | 287 ± 14 | 270 ± 11 | — |
| M21R | RIGHT | RIGHT | 234 ± 6 | 280 ± 5 | * |
| M18S | RIGHT | RIGHT | 295 ± 11 | 268 ± 13 | — |
| M18T | RIGHT | LEFT | 306 ± 14 | 330 ± 19 | — |

* P < 0.05
** P < 0.01

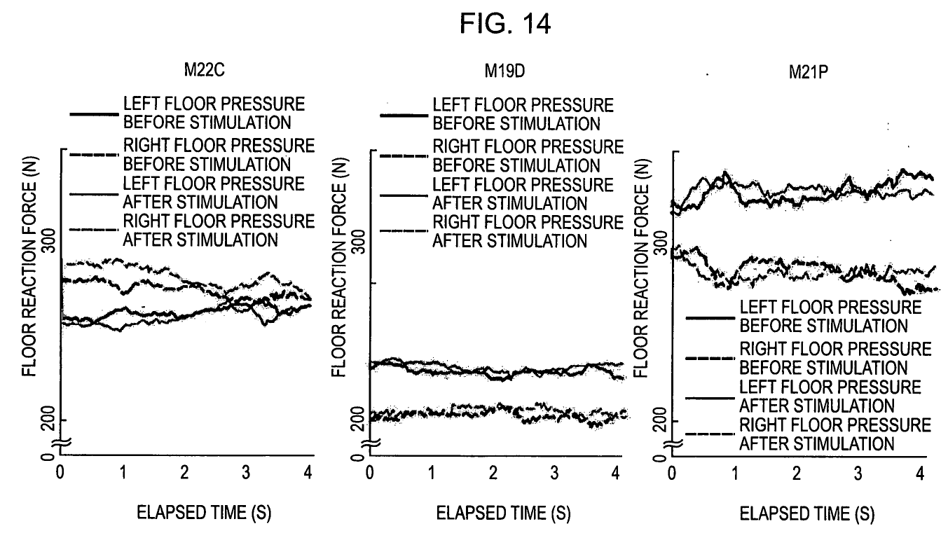

Table 5 shows the collection of the marker positions of the shoulder and the hip, and the significant differences of lateral force reaction force before and after the acupuncture stimulation shown in Table 1 to Table 4 for each subject. 14 men from 20 men had a significant lateral difference of the marker position of the shoulder in the basis posture. 2 men had a risen right shoulder, and 12 men had a lowered right shoulder. That is, all ping-pong players had a slant between the positions of the left and right shoulders in the basic posture. 13 men had a significant lateral difference in the marker position of the hip. 6 men had a risen right hip, and 7 men had a lowered right hip. That is, all of them had a slant between the positions of the left and right hips in the basic posture. Only 2 men did not have the lateral difference in the markers of the shoulder and the hip. Accordingly, it was considered that the basic posture of the ping-pong player had the slant in both or one of the shoulder and the hip. After the acupuncture stimulation, a change in slant was observed. That is, it was observed that the acupuncture stimulation caused the change in posture, for example, the lateral difference of the shoulder or hip, which was viewed before the stimulation, disappeared, or the significant difference was recognized. 4 men had a significant lateral difference in floor reaction force in the basic posture, together with the slant of the position of the shoulder or hip. For 4 men, after the acupuncture stimulation, the significant lateral difference of floor reaction force disappeared. In addition, for 3 men, a significant difference newly appeared. This indicates that the acupuncture stimulation causes the change in center movement. After the acupuncture stimulation, from 8 men having the change in the basic posture, 3 men of M22C, M19D, and M21P had a significant change in floor reaction force. Traces of floor reaction force for 3 men when a shoulder joint is bent are shown in FIG. 14.

TABLE 5
Lateral differences of markers of shoulder and hip, and lateral differences of
floor reaction force in basic posture before and after acupuncture treatment
(list of significant differences for the individual subject of Table 1 to Table 4)

| | | | | | | | TABLE 3 | TABLE 4 |
|---|---|---|---|---|---|---|---|---|
| | | | | | | | BEFORE | AFTER |
| | | | TABLE 1 | | TABLE 2 | | ACUPUNCTURE | ACUPUNCTURE |
| | | | BEFORE | | AFTER | | STIMULATION | STIMULATION |
| | | | ACUPUNCTURE | | ACUPUNCTURE | | FLOOR | FLOOR |
| | DOMINANT | DOMINANT | STIMULATION | | STIMULATION | | REACTION | REACTION |
| SUBJECT | HAND | LEG | SHOULDER | HIP | SHOULDER | HIP | FORCE | FORCE |
| M18A | RIGHT | RIGHT | — | ** | — | * | — | — |
| M18B | RIGHT | RIGHT | — | * | * | * | — | — |
| M22C | RIGHT | RIGHT | ** | *** | * | — | * | — |
| M19D | LEFT | RIGHT | * | *** | — | ** | * | — |
| M18E | RIGHT | LEFT | * | — | * | ** | — | — |
| M20F | RIGHT | RIGHT | — | * | * | *** | — | — |
| M21G | RIGHT | RIGHT | * | ** | * | ** | — | — |
| M19H | RIGHT | RIGHT | ** | — | ** | — | — | — |
| M21I | RIGHT | LEFT | ** | * | *** | *** | — | — |
| M21J | RIGHT | RIGHT | ** | — | ** | — | * | — |
| M18K | RIGHT | LEFT | * | * | * | — | — | — |
| M18L | RIGHT | RIGHT | * | — | ** | * | — | — |
| M20M | RIGHT | LEFT | *** | ** | ** | * | — | — |
| M20N | LEFT | RIGHT | — | — | — | — | — | — |
| M20O | RIGHT | RIGHT | * | * | *** | ** | — | ** |
| M21P | RIGHT | LEFT | * | * | — | — | — | * |
| M20Q | RIGHT | LEFT | * | *** | * | ** | — | — |
| M21R | RIGHT | RIGHT | *** | — | *** | — | — | * |
| M18S | RIGHT | RIGHT | — | — | — | — | — | — |
| M18T | RIGHT | LEFT | — | ** | — | * | * | — |

* P < 0.05
** P < 0.01
*** P < 0.001

Like M19D, there was an example in which an initial lateral difference was maintained even after the acupuncture stimulation. Further, like M22C or M21P, there was an example in which the lateral difference of floor reaction force is expanded due to the bent shoulder joint, and the lateral difference is decreased as the motion ends. It was considered that the marker position of the shoulder or hip in the basic posture or the lateral difference of floor reaction force was influenced by one acupuncture stimulation.

In the present experiment, an influence by the functional slant, such as a dominant hand or leg, and the repetition of the same motion for a long time on the basic posture of the person had been examined through a three-dimensional measurement for the ping-pong players having the career of 7 years or more. As a result, for the ping-pong players, the lateral difference of the marker position of the shoulder or hip in the basic posture or the lateral difference of floor reaction force was recognized, and thus it was considered that the repetition of the same motion for a long time had influence on the posture. The acupuncture stimulation had influence on the slant, and thus the significant difference disappeared or the new significant difference occurred. It was considered that only one acupuncture stimulation had influence on the lateral difference of the marker position of the shoulder or hip in the basic posture or the lateral difference of floor reaction force. Further, with the observation of the trace of floor reaction force from the start of shoulder joint bending to the end in the example in which the acupuncture stimulation causes the change in the basic posture and floor reaction force, the acupuncture stimulation had influence on the entire trace. It was considered that the acupuncture stimulation had influence on the movement of the person.

According to the medical treatment position selection device 500 of the present embodiment, the load motion instructing unit 11 (or the load motion instructing unit 101) can cause the living body to perform the load motion, the measuring unit 100 can store the left and right weight measurement results for the load motion, and the medical treatment position instructing unit 12 can specify the medical treatment position. Further, the measuring unit 100 can notice the time-variant changes in weight of the left and right weight measurement results before and after the stimulation to the medical treatment position.

Further, according to the medical treatment position selection device 500 of the present embodiment, the load motion instructing unit 11 (or the load motion instructing unit 101) can instruct the load motion, the measuring unit 100 can store the left and right weight measurement results for the load motion, and the medical treatment position instructing unit 12 can specify the medical treatment position. Further, the measuring unit 100 can compare the time-variant changes in weight of the left and right weight measurement results for the load motion before and after the stimulation to the medical treatment position, and, when there is a difference, can notice the result as the stimulation result.

### Other Embodiments

The medical treatment position selection device 10 or 500 of the above-described embodiment preferably has a load motion type input unit that, when a predetermined feeling occurs in the living body, inputs a type of a specified load motion.

Further, in the medical treatment position selection device 10 or 500 of the above-described embodiment, it is preferable that, when the predetermined feeling occurs in the living body, the load motion type input unit select and input a degree of the predetermined feeling in the living body according to the type of the specified load motion.

Further, the medical treatment position selection device 10 or 500 of the above-described embodiment preferably has a load motion degree notice unit that, when the predetermined feeling occurs in the living body, digitizes the degree of the predetermined feeling in the living body according to the type of the specified load motion and notices the digitized degree of the predetermined feeling in advance.

Further, the medical treatment position selection device 10 or 500 of the above-described embodiment preferably has a load motion degree input unit that, when the predetermined feeling occurs in the living body, inputs the degree of the predetermined feeling in the living body according to the type of the specified load motion.

Further, the medical treatment position selection device 10 or 500 of the above-described embodiment preferably has a load motion degree selection and input unit that, when the predetermined feeling occurs in the living body, selects and inputs the degree of the predetermined feeling in the living body according to the type of the specified load motion.

Further, the medical treatment position selection device 10 or 500 of the above-described embodiment preferably has a load motion type storing unit that, when the predetermined feeling occurs in the living body, stores the type of the specified load motion.

Further, the medical treatment position selection device 10 or 500 of the above-described embodiment preferably has a load motion type storage data notice unit that, when the predetermined feeling occurs in the living body, reads out and notices the type of the specified load motion from the load motion type storing unit.

Further, the medical treatment position selection device 10 or 500 of the above-described embodiment preferably has a load motion degree storing unit that, when the predetermined feeling occurs in the living body, stores the degree of the predetermined feeling in the living body according to the type of the specified load motion.

Further, the medical treatment position selection device 10 or 500 of the above-described embodiment preferably has a load motion degree storage data notice unit that, when the predetermined feeling occurs in the living body, reads out and notices a degree of the specified load motion from the load motion degree storing unit.

Further, in the medical treatment position selection device 10 or 500 of the above-described embodiment, it is preferable that, when the type of the specified load motion is read out from the load motion type storing unit and is noticed, the degree of the specified load motion be read out from the load motion degree storing unit and is noticed from the load motion degree storage data notice unit in the same unit of notice.

Further, the medical treatment position selection device 10 or 500 of the above-described embodiment preferably has an identification (ID) input unit, with which a person specifies himself to select the medical treatment position. In this case, a plurality of load motion type storage data and load motion degree storage data for plural types of load motions are input to the load motion type storing unit and a load motion degree storing unit in one unit of measurement and are stored therein in at least one unit of measurement.

Further, the medical treatment position selection device 10 or 500 of the above-described embodiment preferably has an ID input unit, with which a person specifies himself to select the medical treatment position, and a medical treatment judging unit that, when a plurality of load motion type storage data and load motion degree storage data for plural types of load motions are set as one unit of measurement, inputs load motion type storage data and load motion degree storage data to the load motion type storing unit and a load motion degree storing unit in different units of measurement, and compares load motion type storage data and load motion degree storage data in different units of measurement.

Further, the medical treatment position selection device 10 or 500 of the above-described embodiment preferably has an ID input unit, with which a person specifies himself to select the medical treatment position. In this case, a plurality of load motion type storage data and load motion degree storage data for plural types of load motions are set as one unit of measurement, and load motion type storage data and load motion degree storage data are input to the load motion type storing unit and a load motion degree storing unit in different units of measurement, and load motion type storage data and load motion degree storage data are individually multiplied in different units of measurement.

Further, the medical treatment position selection device 10 or 500 of the above-described embodiment preferably has an ID input unit, with which a person specifies himself to select the medical treatment position. In this case, a plurality of load motion type storage data and load motion degree storage data for plural types of load motions are set as one unit of measurement, and load motion type storage data and load motion degree storage data are input to the load motion type storing unit and a load motion degree storing unit in different units of measurement, and a sequence of the medical treatment positions associated with the individual load motions is stored.

Further, the medical treatment position selection device 10 or 500 of the above-described embodiment preferably has an ID input unit, with which a person specifies himself to select the medical treatment position. In this case, a plurality of load motion type storage data and load motion degree storage data for plural types of load motions are set as one unit of measurement, and load motion type storage data and load motion degree storage data are input to the load motion type storing unit and a load motion degree storing unit in different units of measurement, and a sequence of medical treatment positions associated with the individual load motions is noticed.

Moreover, the technical scope of the invention is not limited to the above-described embodiments, but various modifications or changes can be made within the scope without departing from the spirit of the invention. Specific materials or configurations in the embodiments are just examples, and can be suitably changed.

What is claimed is:

1. A medical treatment position selection device comprising:
a load motion instructing unit that instructs a load motion as a movement of a living body; and
a medical treatment position instructing unit that instructs a medical treatment position corresponding to the load motion instructed by the load motion instructing unit.

2. The medical treatment position selection device according to claim 1,
wherein the load motion instructing unit instructs plural types of load motions, and
the medical treatment position instructing unit instructs medical treatment positions associated with the individual load motions.

3. The medical treatment position selection device according to claim 2, further comprising:
an input unit that specifies one of the plural types of load motions,
wherein the medical treatment position instructing unit instructs the medical treatment position associated with the load motion specified by the input unit.

4. The medical treatment position selection device according to claim 3,
wherein, when a predetermined feeling occurs in a living body due to the load motion, the input unit specifies the load motion.

5. The medical treatment position selection device according to claim 1, further comprising:
a load motion type input unit that, when a predetermined feeling occurs in the living body, inputs a type of a specified load motion.

6. The medical treatment position selection device according to claim 1, further comprising:
a load motion degree notice unit that, when a predetermined feeling occurs in the living body, digitizes a degree of a predetermined feeling in the living body according to a type of a specified load motion and notices the digitized degree of the predetermined feeling in the living body in advance.

7. The medical treatment position selection device according to claim 1, further comprising:
a load motion degree input unit that, when a predetermined feeling occurs in the living body, inputs a degree of the predetermined feeling in the living body according to a type of a specified load motion.

8. The medical treatment position selection device according to claim 1, further comprising:
a load motion type storing unit that, when a predetermined feeling occurs in the living body, stores a type of a specified load motion.

9. The medical treatment position selection device according to claim 8, further comprising:
a load motion type storage data notice unit that, when the predetermined feeling occurs in the living body, reads out the type of the specified load motion from the load motion type storing unit and notices the type of the specified load motion.

10. The medical treatment position selection device according to claim 1, further comprising:
a load motion degree storing unit that, when a predetermined feeling occurs in the living body, stores a degree of the predetermined feeling in the living body according to a type of a specified load motion.

11. The medical treatment position selection device according to claim 10, further comprising:
a load motion degree storage data notice unit that, when the predetermined feeling occurs in the living body, reads out the degree of the specified load motion from the load motion degree storing unit and notices the degree of the specified load motion.

12. The medical treatment position selection device according to claim 11,
wherein, when the type of the specified load motion is read out from the load motion type storing unit and is noticed, the degree of the specified load motion is read out from the load motion degree storing unit and is noticed from the load motion degree storage data notice unit in the same unit of notice.

13. The medical treatment position selection device according to claim 9, further comprising:
an identification (ID) input unit, with which a person specifies himself to select the medical treatment position,
wherein a plurality of load motion type storage data and load motion degree storage data for plural types of load motions are input to the load motion type storing unit and a load motion degree storing unit in one unit of measurement and are stored therein in at least one unit of measurement.

14. The medical treatment position selection device according to claim 9, further comprising:
an ID input unit, with which a person specifies himself to select the medical treatment position; and
a medical treatment judging unit that, when a plurality of load motion type storage data and load motion degree storage data for plural types of load motions are set as one unit of measurement, inputs load motion type storage data and load motion degree storage data to the load motion type storing unit and a load motion degree storing unit in different units of measurement, and compares load motion type storage data and load motion degree storage data in different units of measurement.

15. The medical treatment position selection device according to claim 9, further comprising:
an ID input unit, with which a person specifies himself to select the medical treatment position,
wherein a plurality of load motion type storage data and load motion degree storage data for plural types of load motions are set as one unit of measurement, and
load motion type storage data and load motion degree storage data are input to the load motion type storing unit and a load motion degree storing unit in different units of measurement, and load motion type storage data and load motion degree storage data are individually multiplied in different units of measurement.

16. The medical treatment position selection device according to claim 9, further comprising:
an ID input unit, with which a person specifies himself to select the medical treatment position,
wherein a plurality of load motion type storage data and load motion degree storage data for plural types of load motions are set as one unit of measurement, and
load motion type storage data and load motion degree storage data are input to the load motion type storing unit and a load motion degree storing unit in different units of measurement, and a sequence of the medical treatment positions associated with the individual load motions is stored.

17. The medical treatment position selection device according to claim 9, further comprising:
an ID input unit, with which a person specifies himself to select the medical treatment position,
wherein a plurality of load motion type storage data and load motion degree storage data for plural types of load motions are set as one unit of measurement, and
load motion type storage data and load motion degree storage data are input to the load motion type storing unit and a load motion degree storing unit in different units of measurement, and a sequence of medical treatment positions associated with the individual load motions is noticed.

18. The medical treatment position selection device according to claim 1,
wherein the medical treatment position instructed by the medical treatment position instructing unit is a therapeutic point.

19. The medical treatment position selection device according to claim 1,
wherein the medical treatment position instructing unit instructs a plurality of medical treatment positions about one load motion.

20. The medical treatment position selection device according to claim 19,
wherein the medical treatment position instructing unit instructs a medical treatment sequence of the plurality of medical treatment positions.

21. The medical treatment position selection device according to claim 1,
wherein the medical treatment position instructing unit has a medical treatment method presenting unit that instructs a medical treatment method against the medical treatment position.

22. The medical treatment position selection device according to claim 1, further comprising:
a measuring unit that measures the living body whose medical treatment position instructed by the medical treatment position instructing unit is stimulated or treated.

23. The medical treatment position selection device according to claim 22,
wherein the measuring unit has a left and right weight measuring unit that individually measures weights of left and right legs in the living body.

24. The medical treatment position selection device according to claim 22,
wherein the measuring unit has a pulse wave measuring unit that measures pulse waves of the living body.

25. The medical treatment position selection device according to claim 22, further comprising:
a medical treatment result judging unit that, on the basis of the measurement result of the measuring unit, judges whether or not an abnormality is reduced in the living body whose medical treatment position is stimulated or treated.
